(19) United States
(12) Patent Application Publication
(10) Pub. No.: US 2025/0090723 A1
(43) Pub. Date: Mar. 20, 2025

(54) TISSUE GRAFT AND PROCESSING METHODS

(71) Applicant: Restoration Biologics LLC, Flagstaff, AZ (US)

(72) Inventors: Suzanne M. TABBAA, Flagstaff, AZ (US); William D. BUGBEE, San Diego, CA (US); Farshid GUILAK, Clayton, MO (US)

(21) Appl. No.: 18/729,369

(22) PCT Filed: Jan. 19, 2023
(86) PCT No.: PCT/US2023/011147

§ 371 Date: Jul. 16, 2024

Related U.S. Application Data
(60) Provisional application No. 63/300,820, filed on Jan. 19, 2022.

(51) Int. Cl. A61L 27/36; A61L 27/22; C12N 9/20

(52) U.S. Cl. CPC A61L 27/3608; A61L 27/227; C12N 9/20; C12Y 301/01003

(57) ABSTRACT

Disclosed herein is a modified tissue graft (120, 200, 300, 500, 630) with improved properties for tissue integration and regeneration. The tissue graft (120, 200, 300, 500, 630) has biological properties that are enhanced by removing lipids (615) and other contaminants (e.g., blood components, micro-organisms) using enzymes, enzyme aggregates, or immobilized enzymes. This application describes the process (10) for removing bio-contaminants while maintaining optimal biological properties.

### CROSS REFERENCE TO RELATED APPLICATIONS

This application claims the benefit of U.S. Provisional Application No. 63/300,820 titled “Improved Tissue Graft and Processing Methods” and filed on Jan. 19, 2022, which is incorporated herein by reference.

### FIELD OF THE INVENTION

The present invention relates to tissue graft and processing methods for autologous tissue grafts or implants used for the hard and soft tissue repair.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Allograft and decellularized tissues are essential adjuncts to tissue repair and regeneration for various clinical applications. Over the past decades, progress has been made in the tissue banking industry to reduce the risk of disease transmission and improve processes to remove biological components that may elicit an immune response or delay tissue repair. Across the hard and soft tissue repair, autologous tissue grafts demonstrate improved biological repair potential and ultimately improved tissue incorporation and outcomes clinically compared to allograft tissues. This has led to a clinical preference for autologous tissues over the inferior performing allograft tissues. The inferior biological properties of allograft tissues can be attributed to the harsh solvents and chemicals utilized to remove the immunogenic portions of the graft. Although current tissue processing technologies are effective at removing immunogenic compounds, the surfactants and chemicals used have been shown to alter the structural properties of the graft, reducing beneficial protein functionality, and leading to localized toxicity as a result of residual cleansing agents. This invention provides improvements to allograft processing by controlling and localizing the removal of bio-contaminants from tissues.

In one application, this technology can be used to improve the bone biological properties of osteochondral grafts (OCG), or bone grafts derived from tissue sources (i.e., animal, human). OCG from human donors are the standard of care for treating large chondral and osteochondral defects. OCG are comprised of both viable cartilage tissue and subchondral bone. Chondrocyte viability is critical to the success of the repair. Limitations of this procedure involve preserving chondrocyte viability and health during prolonged storage and failures associated with subchondral bone incorporation. The poor bone incorporation and remodeling may be attributed to contaminants in the marrow housed within the bone. Contaminants of tissues include lipids, allogeneic cells, and other immunogenic elements. Lipids have been shown to adversely affect bone incorporation. Enzymes may be used to remove immunogenic compounds and modify allograft tissues. Specifically, lipid removal from bone using lipase may be employed. Lipases are enzymes that catalyze the hydrolysis of triglycerides. The use of enzymes to remove immunogenic compounds and modify or decellularize tissues is described in the literature. Specifically, lipid removal from bone tissue using lipase has been described in earlier works (Prior art Tabbaa et al. US 2017/0258962 A1, incorporated herein by reference). The removal of lipids using various detergents and solvents is also well established in the bone grafting literature. Due to the necessity to maintain cartilage health and chondrocyte viability, the use of chemicals and detergents to treat the bone are not used for osteochondral allografts. Processing methods and reagents used to modify the bone must not influence cartilage health or chondrocyte viability. Additionally, bone grafts such as allograft cancellous chips processed with traditional chemicals and surfactants to remove lipids can alter the matrix architecture and limit the bone forming properties of the tissue. This invention describes a tissue graft, such as an osteochondral graft or bone graft, with enhance properties. This invention provides improvements to contaminant removal approaches by controlling and localizing the treatment to the tissue.

### SUMMARY OF THE INVENTION

This invention describes a tissue graft, such as an osteochondral graft or bone graft, with enhanced properties. This invention provides improvements to contaminant removal approaches by controlling and localizing the treatment to the tissue. Disclosed herein is a modified tissue graft with improved properties for tissue integration and regeneration. The tissue graft has biological properties that are enhanced by removing lipids and other contaminants (e.g., blood components, micro-organisms) using enzymes, enzyme aggregates, or immobilized enzymes. This application describes the process for removing bio-contaminants while maintaining optimal biological properties.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 is a flow chart showing an overall method for processing tissue grafts.

FIG. 2 depicts an embodiment of a method for clearing enzymes following treatment of an allograft.

FIG. 3 shows an osteochondral graft comprising one or multiple peripheral or full-length grooves to improve treatment penetration and bony integration.

FIG. 4 depicts an embodiment of a configuration of grooves cut into the condyles.

FIG. 5 shows an embodiment of a cylindrical plug from the parent allograft.

FIG. 6 shows a side surface of the plug of FIG. 5 with grooves.

FIG. 7 shows a top surface of the plug of FIG. 5 with grooves.

FIG. 8 shows a side view of the plug of FIG. 5 after implantation with grooves that deform together into the defect space enhancing potential graft integration.

FIG. 9 depicts embodiments of pre-operative and intra-operative processing steps for osteochondral tissues.

### DETAILED DESCRIPTION

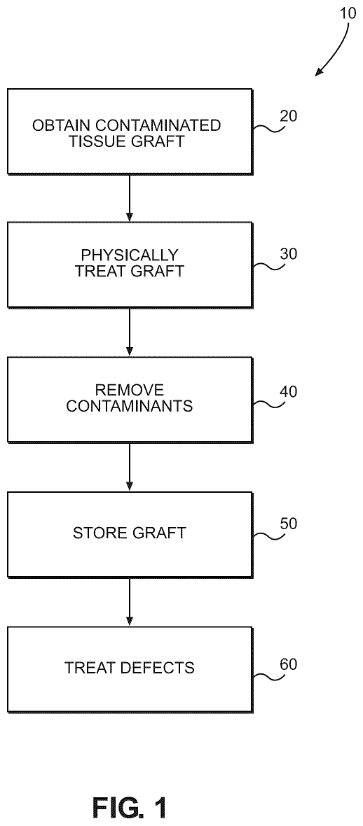

Embodiments of aspects of the present invention relate to providing an enhanced tissue graft by removing contaminants. As shown in FIG. 1, there is a flow chart showing an overall method 10 for processing tissue grafts. Tissue grafts are sourced, at 20, from any tissues derived from human or animals (e.g., allograft, xenograft, autograft). Tissues can include any tissue type or organ include but not limited to musculoskeletal, neural, dermal, cardiovascular, ocular, adipose, etc. Contaminants can include marrow contents, blood vessels, neural tissue, adipocytes, allogeneic cells or cells that can cause an immune response (e.g. immunogenic cells or blood cells), lipids, blood components, proteins, microorganisms (e.g. viruses, bacteria) or components of microorganisms (viral mRNA). The tissue graft is then physically treated, at 30, and then contaminants are removed at 40 to create a tissue graft formed of enhanced tissues. The graft is then stored, at 50, until implanted to treat defects, at 60. Enhanced tissues are those having all contaminants removed, substantially all contaminants removed or sufficient removal wherein remaining contaminants are below a clinically significant level (e.g., remove about 80%, about 78%, about 90%, about 80-85%, about 85-90%, above 90%, etc.). In one aspect, the major contaminants are any type of lipid or lipid-containing cell (adipocyte). Enhanced tissues are those having all lipids removed, substantially all lipids removed or sufficient lipid removal wherein remaining lipids are below a clinically significant level (e.g., remove about 80%, about 78%, about 90%, about 80-85%, about 85-90%, above 90%, etc.). In one aspect, lipids are removed from a bone tissue, osteochondral tissue, or tendon tissue grafts to a reduced level wherein later in-growth or incorporation into a mammalian subject proceeds without impediment or with reduced impediment than if more lipids were present.

The removal of contaminants, at 40, is performed in many different ways. In one specific embodiment, the above levels of lipid reduction are performed using one or more steps of various methods including cross-linked enzyme aggregates with and/or without magnetic particles incorporation to aid in lipid removal. Additionally, enzymes in liquid formulation or immobilized enzymes is used to remove contaminants from allograft tissue. In another embodiment, lipids and other contaminants are reduced using a combination of various types of lipolytic enzymes in aqueous, aggregate, or immobilized forms. In another embodiment, lipids and other contaminants are reduced using a specific lipolytic enzyme type in optimal conditions. Additionally or optionally, the above mentioned reduced level of lipids is accomplished using one or more steps in a method of a localized approached to remove lipids including exposure to one or more steps using high frequency ultra sound, Additionally or optionally, the above mentioned reduced level of lipids is accomplished using one or more steps in a method of a cryoablation process. Additionally or optionally, the above mentioned reduced level of lipids is accomplished using one or more steps in a method of a process of exposure to a form of CO2. The bony portion of the OCA is also preferably enhanced, again at 30, through formation of either peripheral grooves or grooves that transverse the condyles to enhance both penetration of cleansing agents and graft insertion and subsequent bone integration. Additionally or optionally, the removal of contaminants is accomplished through the formation of channels or grooves using a water cutter or other techniques. Additionally or optionally, the cleansing can take place in specific segments of the bone allowing for cartilage to be preserved. Additionally, or optionally, the cleansing partially demineralizes the surface of the bone and expose underlying proteins and growth factors in the native collagen matrix.

### Tissue (e.g. Allograft or Xenograft) Enhancement Using Enzymes Alone or in Combination

Tissue grafts can be sourced from animal or human tissues. Tissue grafts include hard or soft tissues. Examples of hard tissues include bone or osteochondral tissue. Examples of soft tissue include meniscus, tendon, cartilage, vessels, heart valves, pericardium, intervertebral disc, nervous tissue, etc.

In one embodiment, an enhance tissue graft is generated through the treatment of a lipases in combination with a phospholipase and/or a protease. In another embodiment, a lipase is combined with phospholipase and a collagenase to enhance a tissue graft. The specificity of the enzyme and combination will vary depending on the type of tissue. In another embodiment, an enhanced osteochondral graft OCG, bone graft, or tendon graft from human or animal sources is formed through the use of non-specific lipases or lipases with positional or fatty acid specificity (e.g., 1,3-specific lipase) with activity between the ranges of hypothermic (e.g., 0-10° C.), room temperature (e.g., 15-30° C.), or body temperature (e.g., 30-40° C.) and pH of 1-15. The lipase is used in aqueous form. The reaction takes place in a glass reactor under continuous shaking. The reaction time can vary between 5 min to 60 min, 1 hr to 72 hours, or 1 day to 14 days. The substrate to enzyme solution ratio range between 1:1 to 1:40. The dose varies by the enzyme activity and amount of substrate. The dose of the enzyme preferably ranges from 0.01% to 99% w/w of the substrate.

The lipase used to be non-specific or specific have positional or fatty acid specificity. Examples of types of lipases include lipase from Candida rugosa, Penicillium chrysogenum, Candida antartica, Aspergillus niger.

### Tissue Enhancement Using Cross-linked Enzyme Aggregates or Immobilization of Enzymes

This application describes various embodiments of formulations for an improved enzyme compound. In various embodiments of the compound, there is provided modifications and improvements to one or more properties of a tissue graft. In one specific embodiment, there is provided an effective method for removing enzymes from a tissue graft following treatment. This application describes the process for improving allograft properties using cross-linked enzyme aggregates (CLEAs) or immobilization of enzymes.

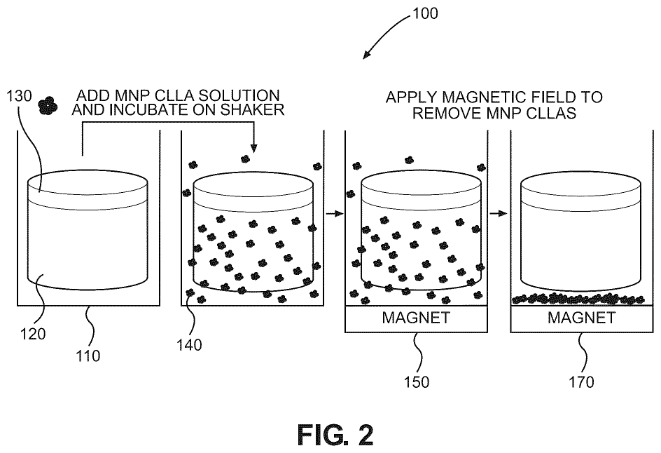

CLEAs contain multiple enzymes that improve the stability and efficiency. The CLEAs used to treat allografts can contain one or multiple types of enzymes including but not limited to lipases, collagenases, phospholipases, proteases, proteinases, phytases, amylases, esterases, cathepsins, matrix metalloproteinases, papain, trypsin, pepsin, amylase, nucleases, colipases, or other enzymes. The CLEAs can be cross-linked with and/or without magnetic particles. The magnetic CLEAs provide effective removal of enzymes from the tissue following treatment using a magnetic field. Earlier inventions describe allograft treatment with enzymes and removal by rinsing with various aqueous solutions. Magnetic CLEAs provides a novel approach to improve recovery of enzymes from tissues, as shown in FIG. 2 which depicts a method of clearing the enzyme after treatment of the allograft as described in detail below. CO₂ provides another effective method for removing broken-down components following treatment with CLEAs due to the molecules ability to bind to non-polar lipid components. Aqueous solutions lack the ability bind to lipid components.

The enzyme aggregates are formed using a traditional immobilizing technique through enzyme precipitation and cross-linking with agents such as glutaraldehyde. Cross-linked aggregates are formed with or without a carrier to improve cross-linking. Examples of carriers include albumin. The magnetic particles can contain iron oxide materials.

Immobilized enzymes are formed by immobilizing one or more enzyme on a carrier which is preferably a natural or synthetic derived matrix. The immobilization can improve stability of the enzyme, a method to remove the enzyme following treatment, and allow for reuse.

### Tissue Graft (E.G., Allograft or Xenograft) Enhancement With Cross-linked Aggregated With and Without Magnetic Particles

The enzyme aggregates are formed using traditional immobilizing techniques. The enzyme aggregates may contain lipases, collagenases, phospholipases, proteases, proteinases, matrix metalloproteinases, phytases, colipase, nucleases, amylases, esterases or a combination of enzymes to treat the bone component. The aggregates may contain lipases, phospholipases, and esterases or other enzymes or a combination of enzymes to remove lipid components or other bio-contaminants.

The aggregates are formed with or without magnetic particles.

### Tissue Graft (E.G., Allograft or Xenograft) Enhancement Using Enzymes in Aqueous Solution or in Aggregate Form With Adjuncts to Improve Removal

In one embodiment, holes, conduits, or grooves are formed in the tissue graft prior to enzyme treatment or washing. The conduits may extend the entire transverse of the tissue or extend partially across the structure. The conduits or grooves can be formed through various methods including water cutting, lasers, drilling, mechanical press, and high frequency ultrasound.

In another embodiment, the conduits or grooves are created through the tissue and aqueous solution containing enzyme formulation is pumped through using a peristaltic pump or similar approach.

In some embodiments, high frequency ultrasound can be localized to various regions of the tissue implant. The graft is placed in a solution conducive to the ultrasound such as buffer or saline solutions. The ultrasound probe is placed near the trabecular bone and pulsed continuously or intermittently to assist in the dissolution of the lipid component of the bone. The frequencies of use range from 10-50 KHz.

In some embodiments, a vacuum system is used to extract lipids, alone or in combination with other treatments.

In some embodiments, cryoablation is used to rupture and remove contaminants. The cryoablation probe is placed into the tissue and the temperature is decreased below 40° C. with intermittent blasts of liquid nitrogen, carbon dioxide, argon, nitrous oxide or other biocompatible cryogens. The tissue is flushed with warm saline or buffer to assist in flushing the lipids from the bone, while the repeated freeze/thaw cycle increases lipid solubility.

In some embodiments, CO₂ is utilized to remove contaminants and lipid contents. Supercritical CO₂ or simply high pressure CO₂ can be used to wash out lipid and other immunogenic compounds from within the cancellous bone of the graft. The high-pressure carbon dioxide is flushed into the tissue and allowed to diffuse. Then the graft is washed with saline or buffer to ensure lipid evacuation from the graft.

It will be appreciated that any part or component of the above described methods of tissue treatment and preparation can be combined with any one or more other parts of components of the above described methods of tissue graft enhancement.

### Osteochondral Graft Enhancement With Cross-linked Enzyme Aggregates With and Without Magnetic Particles

In one embodiment, the cross-linked enzyme aggregates with a size range between (6 nm-50 μm) containing 1 to 50,000 enzymes per aggregate to treat biphasic viable osteochondral grafts from tissue sources (e.g., human or animal). Enzyme aggregate sizes are optimized to be small enough to diffuse into the bone, but also large enough to not affect the cartilage matrix. The enzyme aggregates are formed using traditional immobilizing techniques. The enzyme aggregates may contain lipases, phospholipases, esterases, collagenases, or a combination of enzymes to treat the bone component. The aggregates may contain lipases, phospholipases, and esterases or a combination to remove lipid components from only the bony component of the osteochondral allograft. The CLEAs are filtered to remove any aggregates <6 nm.

Aggregates contain lipase, phospholipases, esterases, proteases, collagenases or a combination of the enzymes that contain optimal activity between the ranges of hypothermic (e.g., 0-10° C.), room temperature (e.g., 15-30° C.), or body temperature (e.g., 30-40° C.).

In another embodiment, the aggregates only contain lipases with optimal activity between 0-10° C.

The aggregates are formed with or without magnetic particles.

### Processing Osteochondral Grafts With Cross-linked Lipase Aggregates With Functionalized Magnetic Nanoparticles (MNP CLLA)

In one embodiment, the osteochondral tissues are procured from donors (e.g., animals, humans). Osteochondral tissues included but not limited to are condyles, patella, talus, elbow, femoral head, carpal or tarsal joints, etc. The whole tissues are washed with various solutions including hypertonic saline containing antibiotics and antioxidants. A processing grafts 100 is shown in FIG. 2. A container 110 filled with solution 120 is used to process the osteochondral tissues 130. Following washing, osteochondral tissues 130 are submerged in closed container 110 containing hypertonic saline solution or culture media 120 (e.g., DMEM without FBS). Antioxidant additives and cross-linked enzyme aggregates with magnetic particles (MNP CLLA) 140 are then added. The solution of MNP CLLA may vary from 0.01%-90% (w/w of solution). The solution is continuously mixed to prevent the MNP CLLA from settling during the incubation period using a shaker, agitator, mixer. The incubation range is from 5 min-72 hours. Following incubation, an external magnetic field is applied to the solution using a magnet 150 to recover >70% (or >80%, or >90%, or >99%) of MNP CLLA, at 170 as shown in FIG. 2. The osteochondral tissue is removed from solution, washed, and placed in a long-term storage container with preservation media to maintain tissue viability.

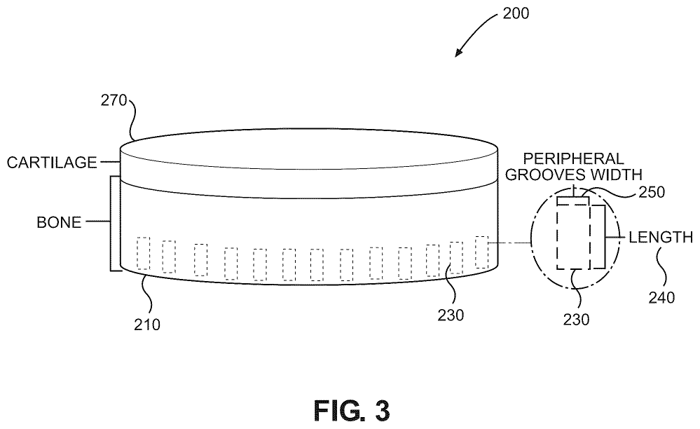

In another embodiment, osteochondral tissue grooves are formed prior to washing and the marrow removal steps. FIG. 3 shows an osteochondral graft 200 having section 210 made of bone and containing one or multiple peripheral or full-length grooves 230 to improve treatment penetration and bony integration. The peripheral or full-length grooves 230 are formed on the distal portion of the bone 210 and have a length 240. The dimensions can embody 10 μm-1 mm in width 250 and 100 μm to the entire length of the condyle 210. The grooves 230 may extend the entire transverse of the bony structure 210 or extend partially across the structure. The grooves 230 can be formed through various methods including water cutting, lasers, drilling, mechanical press, and high frequency ultrasound. A cartilage layer 270 is formed on bone 210.

In another embodiment, the conduits or grooves are created through the bone of the implant and aqueous solution containing enzyme formulation is pumped through using a peristaltic pump or similar approach.

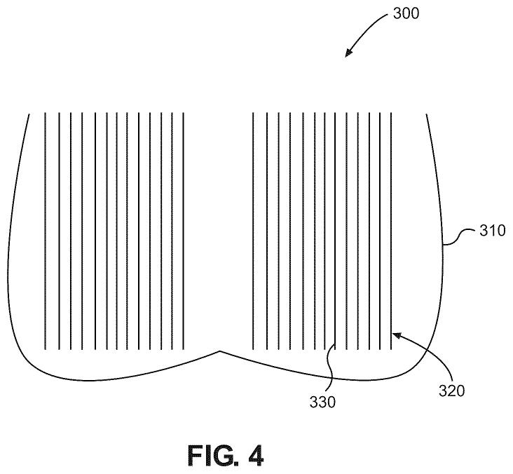

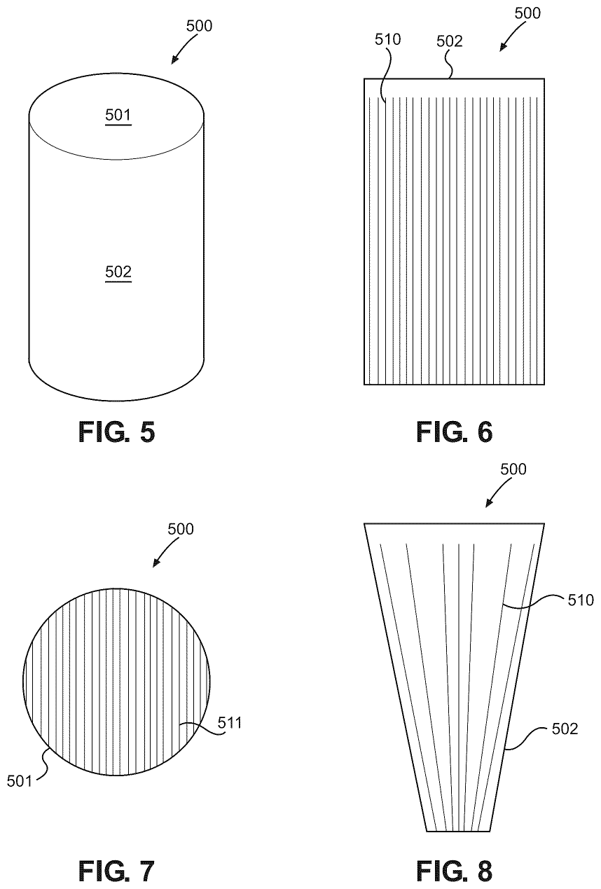

In some embodiments, as shown in FIG. 4 a graft 300 with an outer surface 310 with grooves 320 has each groove 330 pre-cut into the condyles prior to donor graft harvesting to allow penetration of reagents and to allow a better press-fit after being harvested to the desired size for the recipient site. As best seen in FIG. 5. a donor plug 500 is preferably formed in the shape of a cylinder with a top surface 501 and a curved side surface 502. As seen in FIG. 6, curved side 502 can comprise some number of grooves 510 and top side 501 can also have grooves 511 from the parent allograft. Grooves 510 allow the donor plug 500 to deform, as best seen in FIG. 8 as it is pressed into a tapered recipient site. This improves the technical case and creates additional pressure on the surrounding tissue, enhancing bone integration and preventing potential graft migration.

Described herein are processing methods that remove lipids from osteochondral allografts without causing damage to the cartilage health—(1) maintain at least 70% chondrocyte viability or 80% viability or >90% viability, (2) maintain normal articular cartilage biomechanics, (3) reduce matrix degradations by maintaining matrix components—proteoglycans, collagen (4) maintain the subchondral bone integrity and protein and matrix structure.

In some embodiments, lipids are removed from osteochondral allografts prior to prolonged storage using high frequency ultrasound that is localized to the bony portion of the graft. The graft is placed in a solution conducive to the ultrasound such as buffer or saline solutions. The ultrasound probe is placed near the trabecular bone and pulsed continuously or intermittently to assist in the dissolution of the lipid component of the bone. The frequencies of use range from 10-50 KHz.

In some embodiments, cryoablation is used to rupture the adipose marrow to readily flush out the lipid components. The cryoablation probe is placed into the trabecular bone and the temperature is decreased below 40° C. with intermittent blasts of liquid nitrogen, carbon dioxide, argon, nitrous oxide or other biocompatible cryogens. The tissue is flushed with warm saline or buffer to assist in flushing the lipids from the bone, while the repeated freeze/thaw cycle increases lipid solubility.

In some embodiments, CO₂ is utilized to remove lipid contents. Supercritical CO₂ or simply high pressure CO₂ can be used to wash out lipid and other immunogenic compounds from within the cancellous bone of the graft. The high-pressure carbon dioxide is flushed into the tissue and allowed to diffuse. Then the graft is then washed with saline or buffer to ensure lipid evacuation from the graft.

In some embodiments, the graft is contained by a mechanical partition that will prevent the exposure of the cartilage to the agents used to remove the lipids from the cancellous bone.

In some embodiments, the graft is contained by a mechanical partition that will prevent the exposure of the cartilage to the agents used to remove the lipids from the cancellous bone.

Pore sizes of 100-400 μm are well established in the literature to promote angiogenesis and osteogenesis. This invention describes the mechanical formation of peripheral conduits of 50-600 μm in diameter to (1) ensure easier access of the lipid removal agents and (2) encourage faster bony integration at the periphery of the graft and provide quicker stability once implanted. The depth of the conduits may range 200 μm to 5 mm and is dependent on the depth of the subchondral bone. The ratio of the depth of the conduit to the depth of the subchondral bone range from 0.02 to 0.5. The conduits may be created using water cutting, drilling, laser, or other mechanical methods.

It will be appreciated that any part or component of the above described methods of allograft treatment and preparation can be combined with any one or more other parts of components of the above described methods of allograft treatment and preparation.

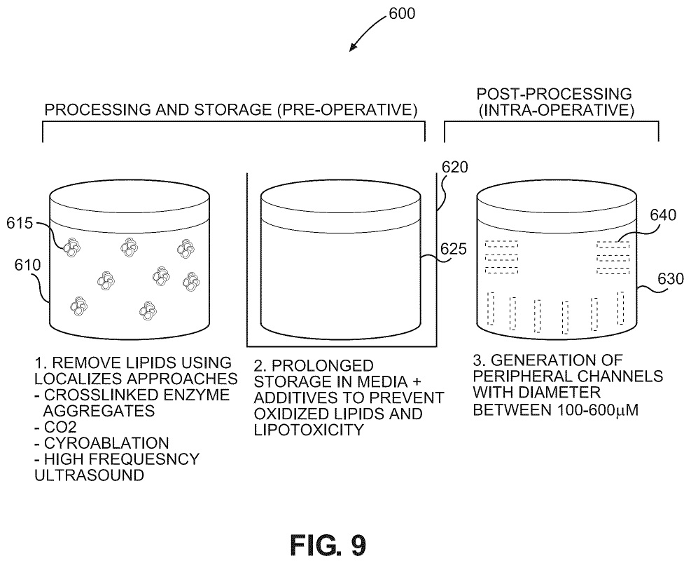

For example, in some embodiments, as shown in FIG. 9, an overall process 600 is described for processing grafts 610. Specifically, the lipids 615 are removed from osteochondral grafts 610 using approaches that are localized to the bone to avoid damage to the cartilage including cross-linked enzyme aggregates, CO₂, cryoablation, and high frequency ultrasound. Following lipid removal, a container 620 is employed to store grafts 625. Container 620 is provided with a media with additives such as antioxidants to prevent the effects of oxidized lipids on the cartilage. Following prolonged storage, the osteochondral grafts may be provided to a surgical center, hospital or other appropriate location for use in treating chondral or osteochondral defects. The graft 630 is altered by the generation of peripheral channels 640 with a diameter of between 100-600 micrometers.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A tissue graft comprising:
tissue with enhanced biological properties and
an amount of contaminants, wherein the contaminants include: marrow contents, blood vessels, neural tissue, adipocytes, allogeneic cells, immunogenic cells, blood cells, lipids, blood components, proteins, microorganisms or viral mRNA, and wherein the amount of contaminants is reduced by 50%, when compared to normal tissue, with enzymes, enzyme aggregates, or immobilized enzymes.

2. The tissue graft of claim 1, wherein the tissue includes:
bone or osteochondral tissue or soft tissues including a least one of meniscus, cartilage, ligament, tendon, bone, pericardium, intervertebral disc, nervous tissue, blood vessels, heart valves, muscle, dermal tissue, and organs.

3. The tissue graft of claim 1 wherein the tissue includes lipids and contaminants below a clinically significant level, with a reduction of at least 70% when compared to normal tissue.

4. The tissue graft of claim 1 wherein the tissue includes channels, grooves, or a modified surface to enhance penetration of an agent.

5. The tissue graft of claim 1 wherein the tissue further includes a bony portion including partially demineralized tissue.

6. The tissue graft of claim 1 wherein the enzymes include a non-specific lipase or 1,3-specific lipase with activity from 0-40° C.

7. The tissue graft of claim 6, wherein a minimum dose of 0.01% of a lipid substrate in the graft is used to treat the graft.

8. The tissue graft of claim 1 wherein the enzymes include a combination of specific enzyme types including lipases, collagenases, phospholipases, proteases, proteinases, matrix metalloproteinases, phytases, colipase, nucleases, amylases, or esterases.

9. A method for removing contaminants from a tissue graft comprising:
obtaining a tissue graft contaminated with one or more of the following contaminates: marrow contents, blood vessels, neural tissue, adipocytes, allogeneic cells, immunogenic cells, blood cells, lipids, blood components, proteins, microorganisms and viral mRNA, and
removing the contaminants with a combination of enzymes, enzyme aggregates, immobilized enzymes, CO₂, or cryoablation.

10. The method of claim 9 comprising applying enzyme aggregates to remove the contaminants.

11. The method of claim 10, comprising applying enzyme aggregates, wherein the enzyme aggregates are cross-linked with and without magnetic particles to facilitate controlled removal of contaminants.

12. The method of claim 10, wherein the tissue graft is a biphasic osteochondral tissue graft, and the method further comprises applying enzyme aggregates, wherein the enzyme aggregates vary in a size range between 6 nm-50 μm and contain 1 to 50,000 enzymes per aggregate.

13. The method of claim 10, comprising applying enzyme combinations or aggregates, wherein enzyme combinations or aggregates contain one more multiple types of enzymes including lipases, collagenases, phospholipases, proteases, proteinases, phytases, amylases, esterases, cathepsins, matrix metalloproteinases, papain, trypsin, pepsin, amylase, nucleases, colipases, or combinations thereof.

14. The method of claim 9, further comprising:
forming tissue modifications including channels, grooves, or perforations.

15. The method of claim 14, wherein the forming the tissue modifications includes drilling or cutting with a water cutter, drill, or laser.

16. The method of claim 9 comprising cryoablation with liquid gas or biocompatible cryogens.

17. The method of claim 9, further comprising:
removing lipid and other contaminants from the tissue using CO₂.

18. The method of claim 9, further comprising employing vacuum extraction with any combination of enzymes, CO₂, or cryoablation.
